(19) United States
(12) Patent Application Publication
(10) Pub. No.: US 2014/0031755 A1
(43) Pub. Date: Jan. 30, 2014

(54) PHOTOLUMINESCENT COUPLING

(71) Applicant: Applied Medical Technology, Inc., Brecksville, OH (US)

(72) Inventors: Derek M. Williams, Cuyahoga Falls, OH (US); Grant W. Phillips, Richfield, OH (US); George J. Picha, Brecksville, OH (US)

(21) Appl. No.: 13/886,457

(22) Filed: May 3, 2013

Related U.S. Application Data
(63) Continuation-in-part of application No. 13/557,850, filed on Jul. 25, 2012.

(51) Int. Cl. A61M 39/10

(52) U.S. Cl. CPC A61M 39/10
USPC 604/175

(57) ABSTRACT

An enteral coupling includes a first input port and a first connector for coupling with the first input port. At least one of the first input port and the first connector has a glow-in-the-dark portion associated with at least a part thereof. The glow-in-the dark portion may comprise only a portion of the device or the entire device at issue. A coupling for use in feeding and/or medicating a patient includes a port, and a connector for coupling with the port. Both the port and the connector are at least in part made of a material having glow-in-the-dark properties.

### CROSS-REFERENCE TO RELATED APPLICATION

This application is a continuation-in-part application of copending U.S. patent application Ser. No. 13/557,850, filed on Jul. 25, 2012, the disclosure of which is incorporated herein by reference in its entirety.

### FIELD

The technology described herein relates to a photoluminescent, or glow-in-the-dark, coupling. In particular, the technology described herein may be used in connection with enteral feeding.

### BACKGROUND

There have been many advancements in the field of gastric feeding devices, such as gastric button devices. Original devices had only one port that provided direct access to a patient's stomach for food and medication administration. With a single port, the use of the device was somewhat simple and straightforward. Newer devices provide two and three ports. For example, newer gastric feeding buttons have a balloon that allows the device to be maintained in the stomach of a user. A port, which is positioned next to the gastric feeding port, is used for introducing water, or in some cases air, into the balloon. More advanced feeding devices, known as Gastric-Jejunal Buttons or (G-J Buttons) have a third port that is used for jejunal feeding. The addition of additional ports provides a greater potential for user error.

Manufacturers have taken steps to design unique characteristics that are used to distinguish the three ports, including dimensional differences, incompatible connections, color differences, printed labels, and indicators molded directly into the devices themselves. Despite all of these steps to ensure that the feeding button is used properly by the end-user, there remains a risk of an accidental connection to an incorrect port. Due to the types of patients that necessitate these particular medical devices, administering food intended for the jejunal port into the gastric port could lead to vomiting and aspiration of stomach contents—a potentially dangerous situation. Thus, care must be taken by the end user to avoid accidental device misuse.

One target patient population for gastric feeding devices is pediatric patients with poor gastric motility. Since these patients will receive most of their nutrition through the jejunal-access port, they will be connected to a feeding pump most of the day and night. Consequently, the patient's caregivers may have to connect and disconnect adapters to the jejunal-access port at night while the patient is asleep. This gives the caregivers two options—they can either turn on a light in order to visually see the device and its different ports, or they can keep the lights off (trying not to disturb the child's sleep) and attempt to make any necessary adjustments to the device by memory and “feel.”

The first option of turning on the light presents a risk of waking the child, who is already in compromised health. The second option deprives the caregiver of the benefits of many of the unique characteristics that are used to distinguish between the three access ports, as described above, and, thus, presents a risk of accidental connection or administration errors.

### SUMMARY

In accordance with the teachings described herein, a coupling for administering food or medication to a patient is described.

### BRIEF DESCRIPTION OF THE DRAWING FIGURES

FIG. 1 is a perspective view of an enteral feeding tube having a first port, a second port, and a third port;

FIG. 2 is a perspective view of the enteral feeding tube of FIG. 1 in a darkened state, showing how one of the ports has an example glow-in-the-dark feature and one of the adapters has a glow-in-the-dark feature;

FIG. 3 is a perspective view of an enteral feeding tube similar to that shown in FIG. 1, but where both the first and second ports have the example glow-in-the-dark feature;

FIG. 4 is a perspective view of the enteral feeding tube of FIG. 3 in a darkened state, showing how two of the ports have an example glow-in-the-dark feature and both adapters have glow-in-the-dark features;

FIG. 5 is a perspective view of a different enteral feeding tube having a first port for feeding and a second port for a balloon;

FIG. 6 is a perspective view of the enteral feeding tube of FIG. 5 in a darkened state, showing how one of the ports has an example glow-in-the-dark feature and an adapter has a glow-in-the-dark feature;

FIG. 7 is an exploded view of the example glow-in-the-dark features of the feeding tube shown in FIGS. 1-4;

FIG. 8 is a cross-sectional view of an example glow-in-the-dark feature applied to a port, where the glow-in-the-dark feature is molded in;

FIG. 9 is a cross-sectional view of an example glow-in-the-dark feature applied to a port, where the glow-in-the-dark feature is externally applied;

FIG. 10 is a top view of an input port with photoluminescent material that has a break in the material;

FIG. 11 is an expanded perspective view of an alternative embodiment of example glow-in-the-dark features applied to ports and connectors within a feeding system;

FIG. 12 is an expanded perspective view of another alternative embodiment of example glow-in-the-dark features applied to ports and connectors within a feeding system; and

FIG. 13 is a perspective view of a feeding system incorporating glow-in-the-dark features.

### DETAILED DESCRIPTION

The technology described herein relates generally to a coupling 10 that has an example glow-in-the-dark or photoluminescent feature 12 that is used to help a user to locate and/or properly orient an adapter or connector 14, 16 with an input port 18, 20 in a dark environment. The example portions of the coupling 10 are generally made of plastic and allow the part to glow-in-the-dark when ambient light is too low to visualize the coupling 10 properly. It is desirable to be able to avoid turning on the lights when a child is sleeping. Thus, the example glow-in-the-dark feature 12 allows a caregiver to access the coupling 10 without turning on the lights. It should be noted that when the term glow-in-the-dark is utilized, it is also meant to encompass photoluminescent and other glow-in-the-dark materials. In addition, when the term photoluminescent is used, it is meant to encompass any number of glow-in-the-dark materials. The terms should be considered to be interchangeable when used herein. In addition, the terms adapter and connector 14, 16 should be considered to be interchangeable when used herein, but can refer to any type of device that may be associated with an opening, a port, or a coupling.

The coupling 10 with the example glow-in-the-dark features 12 may include a glow-in-the dark input port 18, 20, 22 and a glow-in-the-dark connector 14, 16 for coupling with the port 18, 20, such as a feeding tubes 24, 26. Other types of devices may also be useful with the examples described herein, including those not limited to enteral use, such as those relating to parenteral and other uses, as will be discussed in greater detail below.

In one example, a coupling 10 includes an input port 18, 20 and a connector 14, 16 for coupling with the input port 18, 20. The input port 18, 20 includes at least a portion that glows-in-the-dark 12. The connector 14, 16 may also include a glow-in-the-dark portion 12 that is meant to couple with the glow-in-the-dark input port 18, 20. The input port 18, 20 and connector 14, 16 glow-in-the-dark portions 12 may be made of the same glow-in-the-dark resins, if desired, or of different glow-in-the-dark resins.

The glow-in-the-dark resins that are used with the example couplings 10 need minimal light exposure to charge because, in general, the couplings 10 will only have limited exposure times to ambient light. Glow-in-the-dark plastic resins should emit enough light to help the caregiver ensure proper coupling of the connector 14, 16 to the input port 18, 20 during low-light conditions. The glow-in-the-dark plastic resins should emit enough light throughout the night without necessitating a “re-charge” and should be biocompatible.

By providing a glow-in-the-dark material 12 that has the above-describe properties, caregivers (either healthcare professionals or parents) may confidently access different ports 18, 20, 22 within a feeding tube 24, 26 in low-level light conditions. Furthermore, as will be discussed below, the example couplings 10 help to prevent dangerous incorrect connections while giving the patient a better chance to sleep through the night uninterrupted.

The types of materials that may be used for the example glow-in-the-dark features 12 are non-limiting, as long as they charge upon exposure to ambient light and they remain charged for an extended period of time, such as 4, 6, 8 or 12 hours. Different glow colors and glow strength may be desirable under certain circumstances. Thus, different types of glow-in-the-dark materials may be useful instead of the use of a single glow in the dark material. Materials that charge quickly are useful, although those that don't charge quickly may also be useful. The expense of the material along with the particular strengths of the material, such as color, glow strength, length to charge, length to remain changed, etc., should be considered when selecting a glow-in-the-dark material. Any number of other materials may be used, as known by those of skill in the art, as well as materials that may be developed in the future.

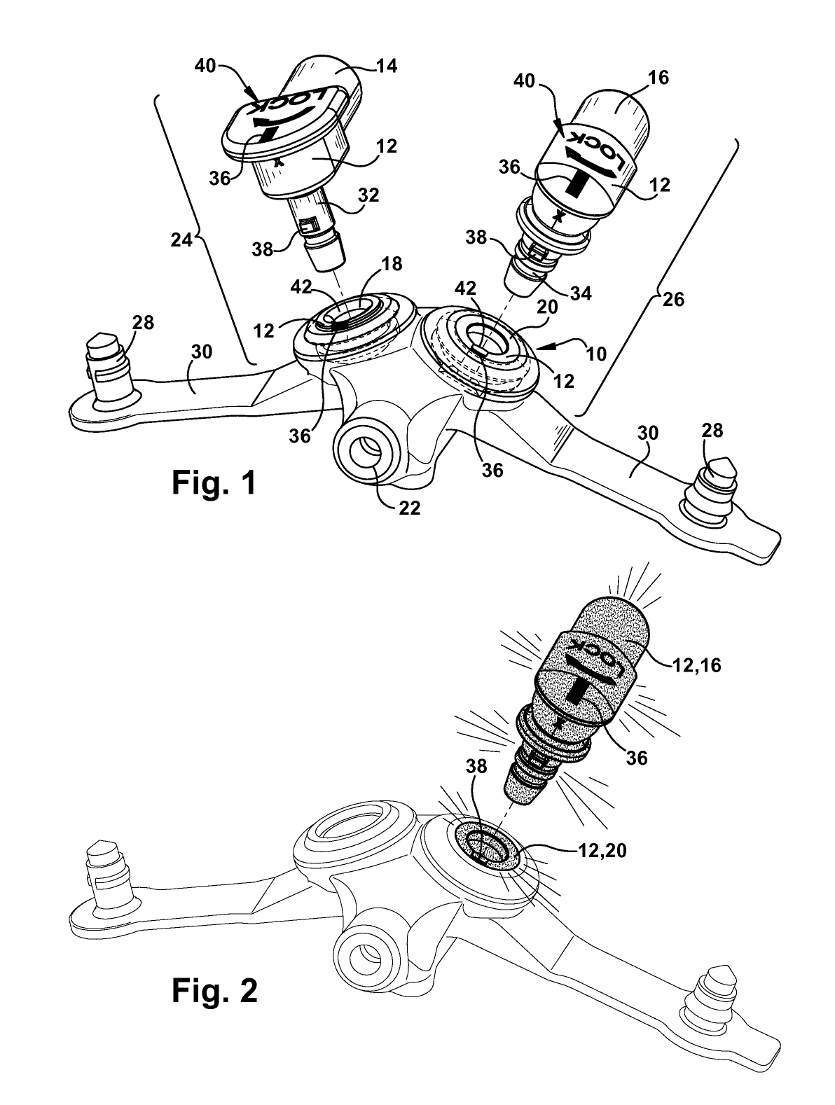

Referring now to the figures, FIGS. 1-4 show an example coupling 10 that is utilized as a button for a G-J tube. This device 10 has an input port 18 for gastric input, an input port 20 for jejunal input, and an input port 22 for filling a balloon with a liquid. The gastric port 18 faces upwardly and is shown on the left side of the button 10. The jejunal port 20 faces upwardly and is shown on the right side of the button 10. The input port 22 for the balloon is positioned on the side of the device 10 and is shown facing forwardly. Both the gastric and jejunal input ports 18, 20 have a plug 28 that is attached to the button 10 with a strip of plastic or silicone material 30, so that when the ports 18, 20 are not coupled to a connector 14, 16, they can be closed by the plug 28. The balloon input port 22 is self-closing. A full description of a G-J button 10 similar to that shown in the examples may be found at www.amtinnovation.com/button_G-JET.html. Similar devices are also discussed in U.S. Pat. Nos. 4,863,438, 5,007,900, 6,019,746, 6,364,858, and 8,048,056.

In the case of the G-J tube, one troubling type of misconnection involves administering feeding intended for the jejunal-access port 20 into the gastric-access port 18. One way to deter this type of mishap under low-light conditions is to only make the jejunal-access port 20 and the jejunal feeding tube coupling adapter 16 glow-in-the-dark. This way the caregiver would be forced to focus on the access port 20 that is glowing-in-the-dark, helping to lessen the possibility of connecting to the incorrect port (which would not be glowing). An example of this is shown in FIGS. 1 and 2.

Another possibility for the G-J tube would be to use two totally different glow-in-the-dark colors for the gastric-access port 18 and the jejunal-access port 20 and connectors 14, 16. If the gastric-access port 18 and its matching adapter 14 was glow-in-the-dark green, for example, while the jejunal-access port 20 and its matching adapter 16 was glow-in-the-dark orange, for example, the caregiver would be able to distinguish both ports 18, 20 in low-level light conditions. Glowing strength or intensity may also be adjusted such that the primary jejunal port 20 glows much brighter than the secondary gastric port 18. Thus, a caregiver could distinguish between the connectors 14, 16 to associate them with the proper port 18, 20.

Importantly, different types of feeding compounds are administered for jejunal feeding as compared to gastric feeding. Thus, it is important that a user does not accidentally administer gastric feeding matter to the jejunal port 20 and vice versa. Such accidental feeding can result in harm to a patient. Thus, it is important to properly label and/or identify the ports 18, 20 so that a user can easily determine which port is used for jejunal feeding and which port is used for gastric feeding.

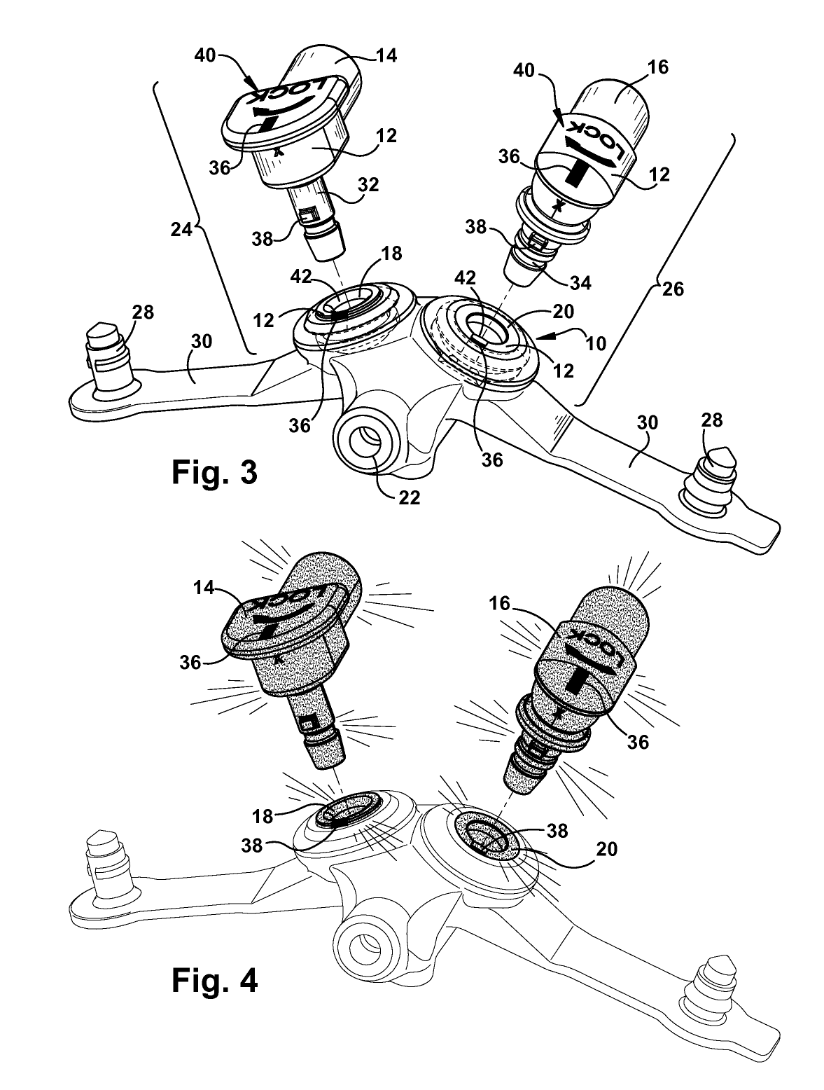

As shown in FIGS. 1 and 3, the jejunal port 20 is positioned on the right side of the coupling 10 and the gastric port 18 is positioned on the left side of the coupling 10. The feeding tube connector 16 that is associated with the jejunal port 20 has a shape that is different from the feeding tube connector 14 that is associated with the gastric port 18. As shown, the head 32 of the connector is smaller for the gastric connector 14 than is the head 34 of the jejunal connector 16. Different shapes may be used as a means for distinguishing the part and/or connectors. The connectors 14, 16 can also be made in different colors or labeled appropriately, if desired. Thus, a caregiver who is familiar with the connectors 14, 16 and their shape can readily determine which connector 14, 16 should be used for jejunal feeding and which connector should be used for gastric feeding.

However, in low light conditions, such as in a bedroom of a patient at night, a caregiver may have difficulty in determining which connector 14, 16 should be used with which port 18, 20. The present example port 18, 20, 22 and connector 14, 16 helps to remedy any difficulty that a caregiver may experience by providing a portion of the input port 18, 20, 22 with a glow-in-the-dark portion 12. As shown in FIGS. 1 and 2, the jejunal port 16 glows in the dark and is formed as an insert that is positioned in the port in order to highlight the port 16. Alternatively, the glow-in-the-dark portion may be coupled to the port in any known manner.

The port 16 alone may be glow-in-the-dark, or the connector 20 may also glow in the dark. In the example of FIG. 2, both the input port 20 and the connector 16 glow-in-the-dark. The port 20 and connector 16 may be the same color. Although helpful to a user, this is not required. The connector 16 may also have labeling provided on a surface of the connector 16 or input port 20. In the example shown, the connector 16 and input port 20 glow-in-the-dark and both have black lines 36 that identify an alignment mark 36 for aligning the connector 16 with a key shape 38 to an input port 20, as well as labeling 40 to assist the user in locking the connector 16 into the port 20. Other labeling could also be used that assists the user in identifying the type of port.

In addition, as shown in FIG. 2, the blackened line 36 of the input port 20 identifies a location for a shape change 42 in the port 20. In the example shown, the port 20 has a key-hole shape 42 for accepting a key or protrusion 38 that is positioned on the connector 16. The key 38 seats within the notch 42 of the input port 20 and allows the connector 16 to be fully inserted into the port 20. Then the connector 16 can be locked in place by rotating the connector head 34 into a locked position such that the key 38 is positioned under a portion of the input port 20. The blackened line 36 on the input port 20 can be created in any known fashion, such as by printing black pad ink over or into the photoluminescent material, or by leaving a break 46 (shown in FIG. 10) in the photoluminescent material, among other known techniques.

FIGS. 3 and 4 concern a device similar to that shown in FIGS. 1 and 2, but in this example, both the gastric and jejunal input ports 18, 20 have a photoluminescent portion 12 and both connectors 14, 16 are photoluminescent 12. While the entire body of the connector is shown as being photoluminescent, portions of the body (not shown) or labeling on the body of the connectors 14, 16 could alternatively be photoluminescent. In FIG. 4, the gastric connector 14 and port 18 both glow in the same color, such as green, while the jejunal input port 20 and connector 16 both glow in the same color that is different from the color of the gastric feeding tube 24, such as orange. Alternatively, the two ports 18, 20 and/or connectors 14, 16 could glow in the same color, but with different intensities, or the two ports 18, 20 and/or connectors 14, 16 could glow in the same color, but have different labeling that is visible in a darkened environment. One or the other of the ports and connectors could be glow-in-the-dark portions.

As with the example shown in FIGS. 1 and 2, the input ports 18, 20 have a key hole 42 or notched shape for accepting a key or protrusion 38 that is positioned on the respective connector 14, 16. Each connector 14, 16 may have an alignment label 32 and each input port 18, 20 may have an alignment feature, such as a blackened line 32, to indicate the orientation of the connector 14, 16 for proper insertion into the input port 18, 20.

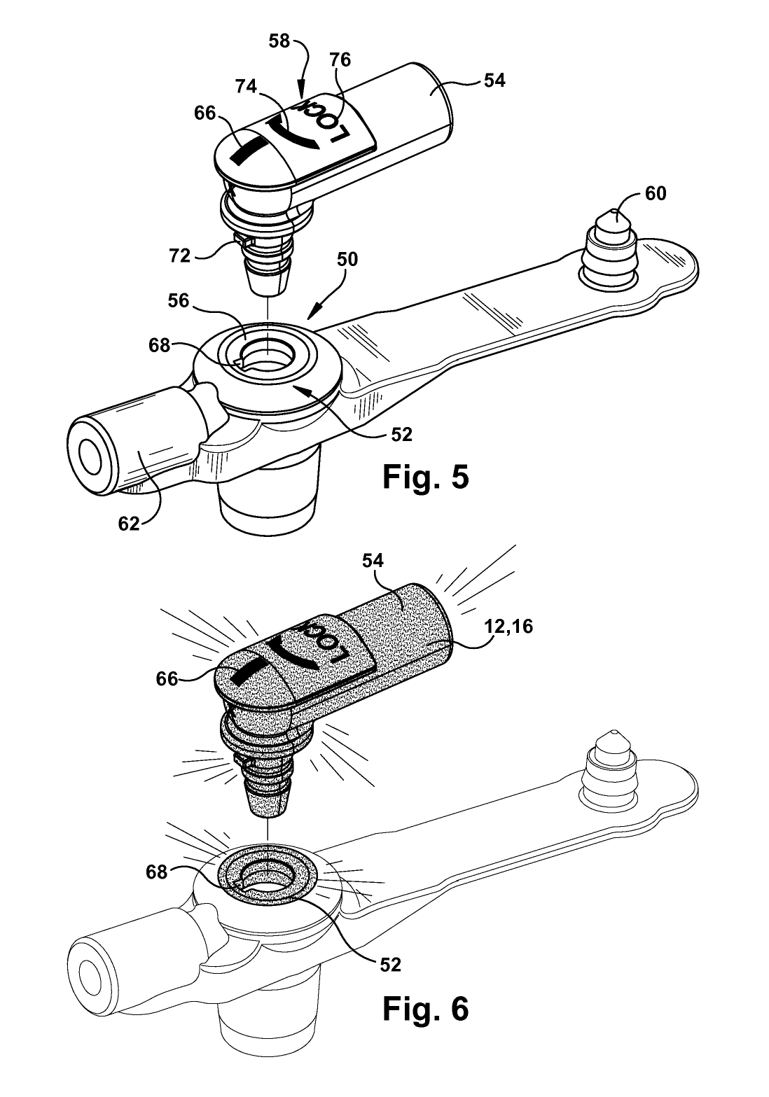

FIGS. 5 and 6 depict a different type of coupling device 50 that has a single input port 52 for food delivery and a balloon access port 62 for filling a balloon in order to maintain the device in position on the body of a patient. Even though the possibility of mixing up the balloon-access port 62 and the feeding-access port 52 is rare, a glow-in-the-dark feeding-access port 52 and connector 54 would assist caregivers during connections and disconnections at night. This would lessen the likelihood of interrupting the sleep of the patient.

In the device 50, shown in FIGS. 5 and 6, the input port 52 for food delivery faces upwardly and is associated with a connector 54. One type of device 50 of this nature that is presently known is the AMT Mini One® Gastrostomy Button 50. In the device shown, the input port 52 has a ring 56 of photoluminescent material that is used to locate the input port 52 in a dark setting. The ring may be coupled with or formed into the button 50 in any known manner.

In addition, the connector 54 may also be made of a photoluminescent material 12 that matches the photoluminescent material 12 of the input port ring 56. Alternatively, the connector 54 may be non-photoluminescent or the connector 54 may be a different color or glow intensity from the photoluminescent material of the input port 52. The input port 52 may have a shape that is conducive to a single orientation for the connector 54, or that only allows one size of connector 54 to be inserted into the port 52. This may assist in preventing accidental connection of the wrong connector. In addition, the connector 54 and/or input port 52 may have labels, such as markings, symbols, or wording, that are used to assist a user in properly orienting the connector 54 relative to the input port 52. In addition, labels 58 may be used to properly identify the port 52 and/or connector 54.

The device shown in FIG. 5 also includes a plug 60 that is connected to the coupling 10 for plugging the input port 52 when the connector 54 is not inserted into the input port 52. In addition, the input port 62 for the balloon is shown extending to the side relative to the coupling 50. The balloon input port 62 shown does not have the same shape or size as the input port 52 for food delivery, making it harder to accidentally administer food through the balloon port 62. In addition, the balloon port 62 shown is self-closing and does not require a plug. Alternatively, a plug could be used if desired or needed.

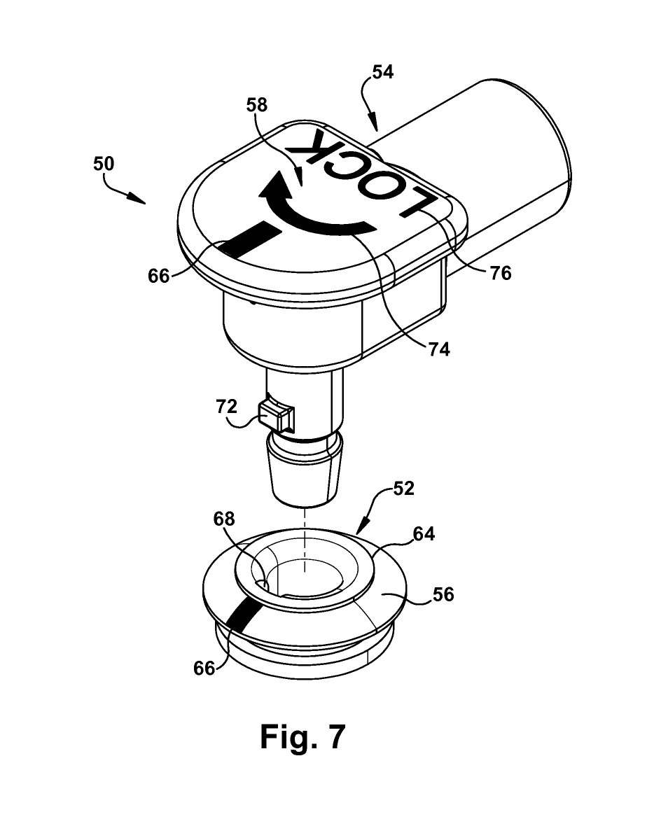

FIG. 7 depicts an exploded view of an input port ring 56 that is coupled to or molded into the coupling 50, as well as a connector 54 for use with the input port ring 56. The input port ring 56 and connector 54 may both be photoluminescent 12. The ring 56 has a lip 64 around a top edge for seating under part of the coupling 50 during the molding process. The ring 56 also has a strip of black ink 66 and a notch 68 that are together used for aligning a connector 54 with the port 52. The strip of black ink 66 is aligned with the notch 68 and is utilized to assist a user in locating the notch 68 for proper orientation of a connector 54. The general shape of the input port 52 is round, except for the notch 68. The black line 66 may be positioned under a clear portion of the coupling 50 so that it is visible when the input port 52 is molded into the coupling 10. The coupling 50 may be made of a transparent material, or of another material if desired. In the event that the coupling 50 is not made of a transparent material, the input port 52 can be formed so that the black line marking 66 is visible. Alternatively, the black line marking 66 can be positioned in a different location, such as on the notch 68 itself, or a different marking can be used.

Instead of a black ink line 66 that is printed or otherwise disposed on the surface of the input port 52, a break 46 in the photoluminescent material 12 of the input port 52 may be used to provide a line for orientation purposes, such as shown in FIG. 10. Other types of markings may alternatively be used, including non-printed markings, stickers and the like.

As shown in FIG. 7, the connector 54 has a key 72 for seating in the input port 52. The key 72 that is used to align the connector 54 with the input port 52 has a shape that allows it to fit within the notch 68. In addition, the connector 54 also has a line printed 66 or otherwise disposed on the surface of the connector 54 for aligning with the notch 68 and which serves as an orientation indicator. The connector 54 also has an arrow 74 and the word “lock” 76 disposed on an upper surface thereof for indicating how the connector 54 may be locked into the input port 52.

The input port 52 may have disposed on a lower surface thereof a ledge 78 that is positioned directly adjacent the notch 68. The ledge 78 is designed to hold the key 72 under the ledge 78 in order to hold the connector 54 in the input port 52. Other types of connection mechanisms may alternatively be utilized. The ledge 78 can be positioned on part of the coupling 50 instead of or in addition to being positioned on the input port ring 52. The connector 54 is designed to prevent leakage from the input port 52 when properly connected. Other indicia or markings may be provided on the connector 54 or on the input port 52 or coupling 50 in order to assist a user in locking, aligning, or otherwise using the connector 54 and input port 52.

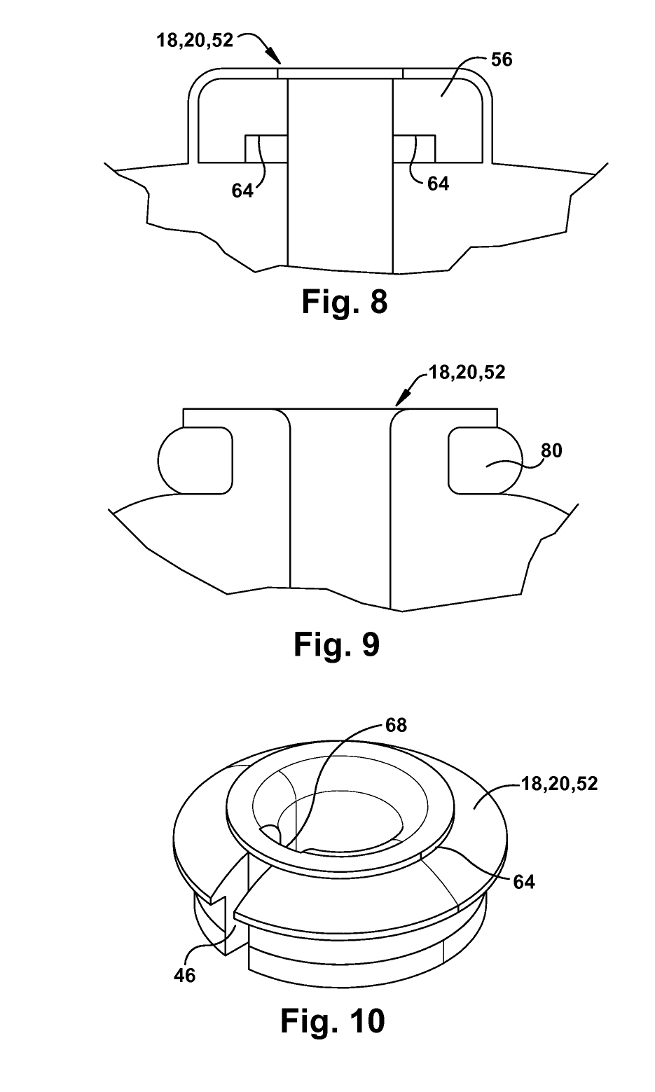

FIGS. 8 and 9 show different types of manufacturing methods for incorporating a photoluminescent material 12 into a coupling 10, 50. FIG. 8 depicts an example of a ring 56 that is molded or adhered/bonded into the port and FIG. 9 depicts an example of an externally applied ring 80. Other types of manufacturing methods and/or designs may alternatively be used. For example, a sticker could be applied to an exterior surface of the coupling 10, 50, or a photoluminescent material could be applied to an exterior surface of the coupling 10, 50. Instead of having the entire connector 14, 16, 54 be made of a photoluminescent material, portions of the connector could be photoluminescent (not shown), or portions of the connector could have photoluminescent materials applied thereto (not shown) in any known manner, such as via printing, sticker, or other means of application.

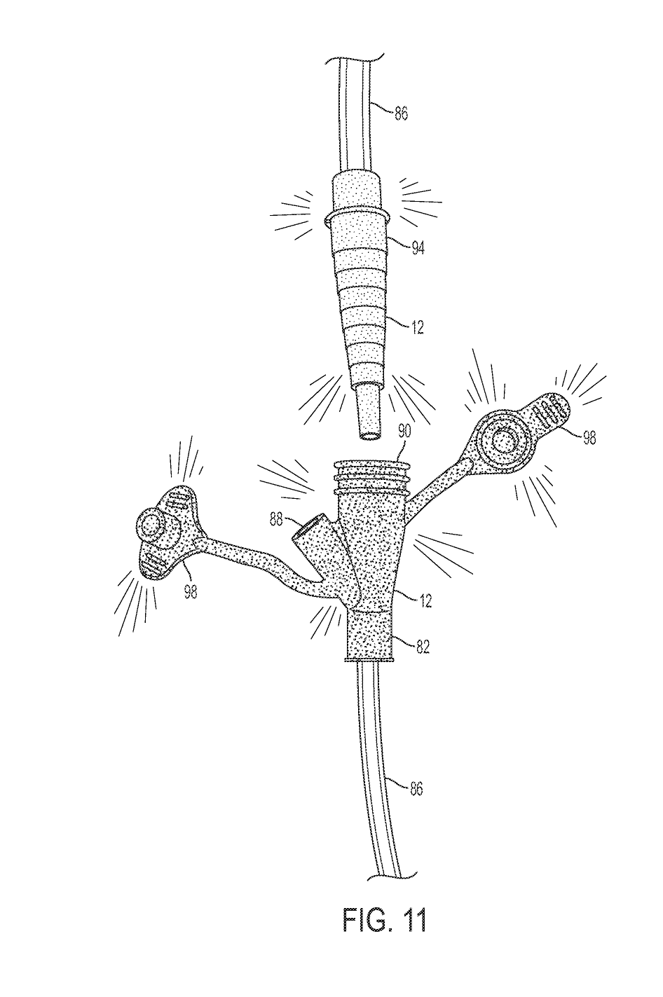

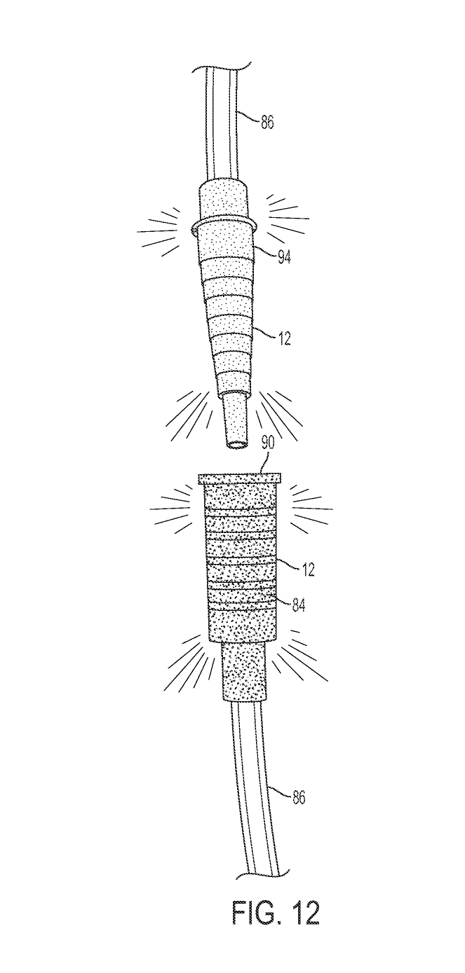

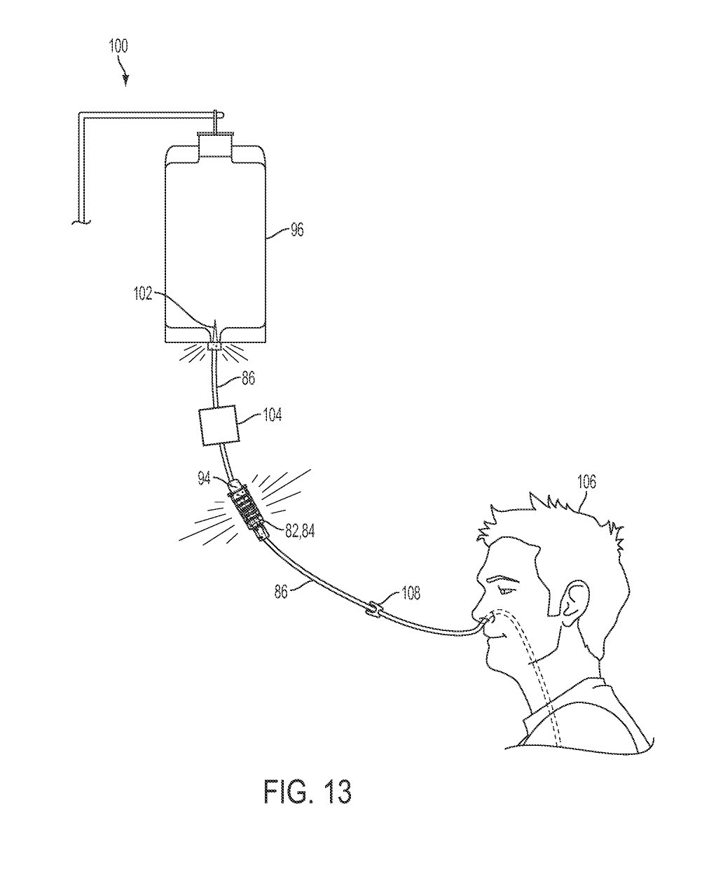

In addition to the ports and connectors described above, other ports and connectors may also utilize glow-in-the-dark characteristics. For example, as shown in FIGS. 11-12, connectors and ports that are spaced a distance from the button 10 may utilize glow-in-the-dark properties in order to aid a caregiver in low-light situations, such as in a bedroom or hospital room of a patient at night. FIGS. 11-12 show different types of connectors, namely Y-port 82 and bolus port 84 connectors located on the opposite end of the feed set tube 86 from connectors 14, 16. It is noted that the connectors 82, 84 are typically not used together, but either a Y-port connector 82 or a bolus port connector 84 is selected to connect to a food source based on caregiver/patient preferences and needs. The Y-port and bolus port connectors 82, 84 are elastomeric food source connectors utilized for coupling directly with a food source, such as a feeding bag (shown best in FIG. 13).

The Y-port and bolus port connectors 82, 84 may be made of a medical-grade, low durometer, highly flexible, PVC resin, but can be any suitable elastomeric or other material. For example, silicone, polyurethane, thermoplastic elastomer, SEBS block copolymers, etc., may be used. Elastomeric materials in these connectors allow for the accommodation of a variety of different coupling possibilities, as well as aiding in the retention force for the coupling. The Y-port and bolus port connectors 82, 84 are examples of the types of ports that utilize a glow-in-the-dark feature. Any rigid connector that can be connected to a food source may alternatively be glow-in-the-dark.

As shown in FIG. 11, the Y-port connector 82 includes a side port 88 and an input port 90. The input port 90 may be for use with a Christmas tree connector 94. The side port 88 can be utilized for injecting medicine into the Y-port connector 82 without having to disconnect the Y-port 82 from the feeding source. The input port 90 is typically utilized for the input of feeding materials. The input port 90 may include ridges 92 to grip a Christmas tree connector 94 that comes directly from a feeding bag 96. Both the side and input ports 88, 90 may utilize a plug or pinch clamp 98 that is attached to an exterior portion of the Y-port connector 82 to stop flow without disconnecting any of the other parts discussed herein. A clamp 108 may also be installed around the tubing 86 below the Y-port connector 82 or bolus port connector 84 in order to stop flow of feeding material or medication to a patient, as shown in FIG. 13.

Similarly, the bolus connector 84 has an input port 90 that is configured to mate with a connector, such as a Christmas tree connector 94 that comes directly from a feeding bag 96, as shown in FIGS. 12-13. The bolus port connector 84 does not utilize a separate side port. Thus, larger amounts of food can typically be supplied through the bolus port connector 84. Medication may be input to the bolus connector 84 by disconnecting the input port 84 from the connector 90. As shown, both the Y-port and bolus port connectors 82, 84 do not have locking or positioning features like the key 38 and key-hole 42 for the coupling 10 on the opposite end of the tube 86 as disclosed herein. However, such features could be incorporated.

As shown in FIG. 13, an example feeding set system 100 comprises a feeding bag 96, a coupling 102 for mating with the feeding bag 96, a Christmas tree connector 94 on the end of the tubing 86 that extends from the feeding bag 96, and a Y-port or bolus port connector 82, 84 having tubing 86 extending therefrom. The tubing 86 below the ports 82, 84 may extend to the feeding button 10, discussed above in connection with FIGS. 1-10, or may extend into the nose of a patient 106 for nasogastric feeding. A pump 104, shown schematically in FIG. 13, may be installed between the Y-port connector 82 or bolus port connector 84 and the spike 102. The pump 104 may be used for controlling the timing and amount of nutrition delivered to a patient 106. As shown, the Christmas tree connector 94 on the end of the tubing 86 connects to the bolus port connector 84 (or Y-port 82) to enable the contents of the feeding bag 96 to reach the patient 106 for nasogastric, gastric, or jejunal feeding, or combinations thereof. The example system may be utilized for other types of feeding, including parental feeding, if desired.

In low light conditions, such as in a bedroom or hospital room of a patient at night, a caregiver may have difficulty locating the Y-port or bolus port connector 82, 84, and Christmas tree connector 94. The present example Y-port or bolus port connector 82, 84, and Christmas tree connector 94 help to remedy any difficulty that a caregiver may experience by providing a portion or all of the Y-port and bolus port connectors 82, 84, and Christmas tree connector 94 with a glow-in-the-dark portion 12. The glow-in-the-dark portion 12 will help a caregiver locate the Y-port or bolus port connector 82, 84 and Christmas tree connector 94, and connect/disconnect feedings without having to unlock the connectors 14, 16 from the coupling 10 positioned at the opposite end of the tube 86. Moreover, glow-in-the-dark Y-port or bolus port connector 82, 84 and Christmas tree connector 94 would assist caregivers during connections and disconnections at night lessening the likelihood of interrupting the sleep of the patient.

The entire Y-port or bolus port connector 82, 84 and Christmas tree connector 94 may glow-in-the-dark. Alternatively, a portion of the connectors 82, 84, and 94 could glow-in-the-dark. For example, a removable glow-in-the-dark ring or band (not shown) could be installed around the various connectors 82, 84, 94, 102 if desired. The glow-in-the-dark portion could be formed around the opening to the connectors 82, 84, 94 in order to make it easier for the caregiver to find the opening of the connectors 82, 84, 94. Alternatively, the glow-in-the-dark portion could be coupled to the connectors in any known manner. In addition, the coupling 102 or spike that couples to the feeding bag 96 could glow-in-the-dark in order to assist in changing feeding bags. The port for the feeding back could glow-in-the-dark in order to make it easier for a caregiver to utilize the spike 102 in order to open the feeding bag. In addition, part or all of the tubing 86 could glow-in-the-dark, if desired.

As shown in FIGS. 11-12, the different stipple patterns of the Y-port or bolus port connectors 82, 84 and Christmas tree connector 94 illustrate that the Y-port or bolus port connector 82, 84 may be a first glow-in-the-dark color, and the Christmas tree connector 94 may be a second glow-in-the-dark color. Alternatively, the Y-port or bolus port connector 82, 84 and the Christmas tree connector 94 may be the same glow-in-the dark color, but Y-port or bolus port connector 82, 84 may have a first glow-in-the-dark intensity and the Christmas tree connector 94 may have a second glow-in-the dark intensity. Glowing strength or intensity may be adjusted such that the Y-port connector 82 or bolus port connector 84 glows much brighter than the Christmas tree connector 94 or vice versa. Thus, a caregiver could distinguish between the Y-port or bolus port connector 82, 84 and a Christmas tree connector 94 more readily.

The glow-in-the-dark features described above are applicable to all of the parts of a gravity feeding set as well as a pump feeding system. In addition, the glow-in-the-dark features are applicable to nasogastric, gastric, and jejunal feeding, either alone or in combinations thereof, or any other types of feeding. Moreover, the glow-in-the-dark features are applicable to other types of couplings used in low light conditions, such as parenteral nutrition as well as other intravenous therapy.

The glow-in-the-dark principles discussed herein may also be applied to other medical devices that require connections and disconnections in low-level light conditions. Nightly rounds are a common practice in hospitals, and patients are often woken up simply because the lights need to be turned on to make adjustments to devices and couplings. Glow-in-the-dark resins could be used in various couplings including intravenous and arterial lines, dialysis connections, Foley catheter connections, chest tubes, or any other type of liquid, gas, or vacuum connection used for patient care. The housings of the couplings can be color coded for different uses, and the dimensions of the glow-in-the-dark connectors can be varied to prevent misconnections. Glow-in-the-dark catheters and inter-lumen catheters can also be made using the above-described technology. This can help healthcare professionals visualize catheter placement in vessels near the surface of the patient's skin simply by turning the lights down low. If the glow-in-the-dark resins can be made to emit enough light, connectors can be made to help illuminate body cavities as well.

According to one example of the invention, an enteral coupling comprises a first input port and a photoluminescent portion associated with the first input port. The first input port is one or more of a gastric port, a jejunal port, or a balloon port. The first connector is for inserting into the first input port and may include a photoluminescent portion. The second input port may have a photoluminescent portion associated with the second input port and a second connector having a photoluminescent portion. The second connector is for inserting into the second input port. A third input port may also be provided with a third device for insertion into the third input port. The third input port and third device may or may not include a photoluminescent portion.

The photoluminescent portion of the first input port may be integrally coupled to the first input port or externally positioned on the first input port. A second photoluminescent portion having a color that is different from a color of the photoluminescent portion of the first input port may be utilized for indicating orientation. A break in the photoluminescent portion of the first input port for indicating orientation, wherein the break is provided by a second photoluminescent portion that has a different color from the photoluminescent portion of the first input port, or by a break in the material of the photoluminescent portion of the first input port, or by a darkened portion associated with the photoluminescent portion of the first input port.

The photoluminescent portion of the first input port may have a first color and the photoluminescent portion of the second input port may have a second color that is different from the first color. The photoluminescent portion of the first port may have a first color, the photoluminescent portion of the first connector may have a second color, the photoluminescent portion of the second input port may have a third color, and the photoluminescent portion of the second connector may have a fourth color. The first and second colors may be the same and the third and fourth colors may be the same. Alternatively, the first and second colors may be different from one another and the third and fourth colors may be different from one another.

The first input port may have an opening with a discontinuous portion for indicating orientation. The discontinuous portion may be provided by a notch, by black ink pad printing, by a break in the photoluminescent material of the first input port, by a blackened portion, or by a combination thereof.

In one example of the invention, the material of the photoluminescent portion of the first input port, second input port, first connector, or second connector charges with minimal light exposure and lasts for 8 or more hours. One or more of the photoluminescent portions may glow brighter than another of the photoluminescent portions.

The various parts of the coupling may include labels that are visible in the dark. The labels may be positioned on the photoluminescent portion of the first input port and may include black ink or another different photoluminescent portion.

The example coupling may include an orientation indicating means, wherein the orientation indicating means includes black ink, a different colored photoluminescent material, labeling, different shapes, darkened portions, or a combination thereof. Alternatively, the photoluminescent portion may be bright enough to illuminate a body cavity.

In another example of the invention, a coupling comprises a first port having a first glow-in-the dark feature and a second port having a second glow-in-the-dark feature. The first glow-in-the dark feature is different from the second glow-in-the-dark feature. The first glow-in-the dark feature may be a first glow-in-the dark color and the second glow-in-the dark feature may be a second glow-in-the dark color. Alternatively, or in addition thereto, the first glow-in-the dark feature may have a first glow-in-the dark intensity and the second glow-in-the dark feature may have a second glow-in-the dark intensity. Alternatively, or in addition thereto, the first glow-in-the dark feature may have a first glow-in-the dark shape and the second glow-in-the dark feature may have a second glow-in-the dark shape. Alternatively, or in addition thereto, the first glow-in-the dark feature may have a first glow-in-the dark labeling and the second glow-in-the dark feature may have a second glow-in-the dark labeling. A combination of any of the foregoing features may be utilized, as desired.

The coupling may be an enteral feeding tube. The coupling may also include a first connector for coupling with the first port and a second connector for coupling with the second port. The first connector may have a first glow-in-the-dark feature and the second connector may have a second glow-in-the-dark feature.

In another example of the invention, a coupling for administering food or medication comprises a first port having a glow-in-the-dark feature and means for orientation that assists a user in properly orienting a connector to be mounted in the first port in proper orientation, and a first connector coupled to the first port. The first connector is guided by the means for orientation in order to properly couple the first connector to the first port. The means for orientating may be one or more of a shape, a glow intensity, labeling, color, a break in the glow-in-the-dark features, texture, a darkened portion, or a combination thereof.

Another example of an enteral coupling includes a first input port and a first connector for coupling with the first input port. At least one of the first input port and the first connector has a glow-in-the-dark portion associated with at least a part thereof. In addition, the glow-in-the dark portion may comprise only a portion of the device or the entire device at issue.

The coupling may include tubing and a coupling for mating with a food source. The tubing may or may not have a glow-in-the dark portion. The coupling is for mating with a food source and may or may not have a glow-in-the-dark portion. The first input port may be one of a gastric button port, a gastric jejunal button port, jejunal button port, a Y-port connector, or a bolus port connector. The first connector may be a connector for coupling with one of said buttons, or a connector for coupling with the Y-port or bolus port connector.

The coupling may also include a second input port and a second connector. One or both of first and second connectors may at least in part glow-in-the-dark and one or both of the first and second ports may at least in part glow-in-the-dark. The first input port may be a gastric button port, a gastric jejunal button port, jejunal button port. The second input port may be a Y-port connector or bolus port connector. The second connector may couple with the Y-port or the bolus port connector.

The glow-in-the-dark portion of the first input port may have a first feature and the glow-in-the-dark portion of the second input port may have a second feature that is different from the first feature, and the feature may be one or both of color and intensity. The glow-in-the-dark portion of the first port may have a first color, the glow-in-the-dark portion of the first connector may have a second color, the glow-in-the-dark portion of the second input port may have a third color, and the glow-in-the-dark portion of the second connector may have a fourth color. The first and second colors may be the same as one another and the third and fourth colors may be the same as one another. The first and second colors may be different from one another and the third and fourth colors may be different from one another.

The material of the glow-in-the-dark portion of one or more of the first input port, second input port, first connector, and second connector may charge with minimal light exposure and lasts for 8 or more hours. At least one of the glow-in-the-dark ports or connectors has one or both of an intensity and a color that is different from another of the glow-in-the-dark ports or connectors.

In another example, a feed set system includes tubing, a first input port, a first connector, and a coupling for mating with a feed bag. At least one of the tubing, the first input port, the first connector, and the coupling are for mating with a feed bag has a glow-in-the-dark property.

The glow-in-the-dark property may have one or both of a glow-in-the-dark color and a glow-in-the-dark intensity. The first input port may be one of a gastric button, a gastric jejunal button, a jejunal button, a Y-port connector, or a bolus port connector. The first connector may be a connector for coupling with the first input port.

A second input port and a second connector may also be included. One or both of the first and second connectors at least in part glow-in-the-dark and one or both of the first and second input ports at least in part glow-in-the-dark. The first input port may be part of a gastric, gastric-jejunal, or jejunal button. The first connector may be a connector for coupling with the first input port. The second input port may be a Y-port connector or a bolus port connector. The second connector may be a Christmas tree connector for coupling with the Y-port or bolus port connector.

The first input port may have a glow-in-the-dark color, the first connector may have a second glow-in-the-dark color, the second input port may have a third glow-in-the-dark color, and the second connector may have a fourth glow-in-the-dark color. The first and second colors may be the same and the third and fourth colors may be the same. The first and second colors may be the same as or different from the third and fourth colors, or the first and second colors may be different from one another and the third and fourth colors are different from one another. The glow-in-the-dark property charges with minimal light exposure and lasts for 8 or more hours.

An alternative example coupling for use in feeding and/or medicating a patient includes a port and a connector for coupling with the port. Both the port and the connector are at least in part made of a material having glow-in-the-dark properties.

The input port and the connector may be associated with enteral or parenteral feeding. The coupling may further comprise tubing coupled to the connector and one or both of a food and a medicine source coupled to the tubing. The entire port and the entire connector have glow-in-the-dark properties.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

1. An enteral coupling comprising:
a first input port; and
a first connector for coupling with the first input port,
wherein at least one of the first input port and the first connector has a glow-in-the-dark portion associated with at least a part thereof, and the glow-in-the dark portion may comprise only a portion of the input port or connector or the entire input port or connector at issue.

2. The coupling of claim 1, further comprising tubing and a coupling for mating with a food source, wherein the tubing may or may not have a glow-in-the dark portion and the coupling for mating with a food source may or may not have a glow-in-the-dark portion.

3. The coupling of claim 1, wherein the first input port is one of a gastric button port, a gastric jejunal button port, jejunal button port, a Y-port connector, or a bolus port connector; and
the first connector is a connector for coupling with one of said buttons, or a connector for coupling with the Y-port or bolus port connector.

4. The coupling of claim 1, further comprising a second input port and a second connector wherein one or both of first and second connectors at least in part glow-in-the-dark and one or both of the first and second ports at least in part glow-in-the-dark.

5. The coupling of claim 4, wherein the first input port is a gastric button port, a gastric jejunal button port, jejunal button port;
the second input port is a Y-port connector or bolus port connector; and
the second connector couples with the Y-port or bolus port connector.

6. The coupling of claim 4, wherein the glow-in-the-dark portion of the first input port has a first feature and the glow-in-the-dark portion of the second input port has a second feature that is different from the first feature, and the feature is one or both of color and intensity.

7. The coupling of claim 4, wherein the glow-in-the-dark portion of the first port has a first color, the glow-in-the-dark portion of the first connector has a second color, the glow-in-the-dark portion of the second input port has a third color, and the glow-in-the-dark portion of the second connector has a fourth color; and
the first and second colors are the same as one another and the third and fourth colors are the same as one another, or
the first and second colors are different from one another and the third and fourth colors are different from one another.

8. The coupling of claim 4, wherein the material of the glow-in-the-dark portion of one or more of the first input port, second input port, first connector, and second connector charges with minimal light exposure and lasts for 8 or more hours.

9. The coupling of claim 4, wherein at least one of the glow-in-the-dark ports or connectors has one or both of an intensity and a color that is different from another of the glow-in-the-dark ports or connectors.

10. A feed set system comprising:
tubing;
a first input port;
a first connector; and
a coupling for mating with a feed bag;
wherein at least one of the tubing, the first input port, the first connector, and the coupling for mating with a feed bag has a glow-in-the-dark property.

11. The feed set system of claim 10, wherein the glow-in-the-dark property has one or both of a glow-in-the-dark color and a glow-in-the-dark intensity.

12. The feed set system of claim 10, wherein the first input port is one of a gastric button, a gastric jejunal button, a jejunal button, a Y-port connector, or a bolus port connector; and
the first connector is a connector for coupling with the first input port.

13. The feed set system of claim 10, further comprising a second input port and a second connector wherein one or both of the first and second connectors at least in part glow-in-the-dark and one or both of the first and second input ports at least in part glow-in-the-dark.

14. The feed set system of claim 13, wherein the first input port is part of a gastric, gastric-jejunal, or jejunal button;
the first connector is a connector for coupling with the first input port;
the second input port is a Y-port connector or a bolus port connector; and
the second connector is a Christmas tree connector for coupling with the Y-port or bolus port connector.

15. The feed set system of claim 13, wherein the first input port has a glow-in-the-dark color, the first connector has a second glow-in-the-dark color, the second input port has a third glow-in-the-dark color, and the second connector has a fourth glow-in-the-dark color, and
the first and second colors are the same and the third and fourth colors are the same, with the first and second colors being the same as or different from the third and fourth colors, or
the first and second colors are different from one another and the third and fourth colors are different from one another.

16. The feed set system of claim 10, wherein the glow-in-the-dark property charges with minimal light exposure and lasts for 8 or more hours.

17. A coupling for use in feeding and/or medicating a patient comprising:
a port; and
a connector for coupling with the port;
wherein both the port and the connector are at least in part made of a material having glow-in-the-dark properties.

18. The coupling of claim 17, wherein the input port and the connector are associated with enteral or parenteral feeding.

19. The coupling of claim 17, further comprising tubing coupled to the connector and one or both of a food and a medicine source coupled to the tubing.

20. The coupling of claim 17, wherein the entire port and the entire connector have glow-in-the-dark properties.
